(19) United States
(12) Patent Application Publication
(10) Pub. No.: US 2026/0174329 A1
(43) Pub. Date: Jun. 25, 2026

(54) CONTEXTUAL REMOTE PATIENT MONITORING SYSTEM

(71) Applicant: Withings, ISSY LES MOULINEAUX (FR)

(72) Inventors: Younes BRINI, ISSY LES MOULINEAUX (FR); Samuel ATTIA, ISSY LES MOULINEAUX (FR)

(21) Appl. No.: 19/423,549

(22) Filed: Dec. 17, 2025

(30) Foreign Application Priority Data
Dec. 20, 2024 (FR) FR2414846

(51) Int. Cl. A61B 5/00; A61B 5/021; G16H 40/67

(52) U.S. Cl. CPC A61B 5/0004; A61B 5/0022; A61B 5/021; A61B 5/742; A61B 5/7475; G16H 40/67; A61B 2562/0247

(57) ABSTRACT

A remote patient monitoring (RPM) system is disclosed that acquires blood pressure (BP) data from a BP monitor and enriches the data with contextual information obtained directly from the user during the measurement session. The BP monitor includes a display and input hardware for presenting questions and receiving predetermined responses that form contextual data associated with the BP data. The enriched BP data is transmitted to a server, which attributes a notification level using a correspondence database linking BP values and contextual data to notification levels or level variations. A remote monitoring interface, executed on a device used by a healthcare professional, displays the BP data in a display mode determined by the attributed notification level. The system reduces false alerts, improves triage efficiency, and provides clinically actionable information for diagnostic assessment in remote-care environments.

### CROSS-REFERENCE TO RELATED APPLICATIONS

This application claims priority to French Patent Application No. 2414846, filed Dec. 20, 2024, the entire content of which is incorporated herein by reference in its entirety.

### FIELD

The invention relates to remote patient monitoring system, known as RPM system. Those systems usually involve a measuring device, such as a BP (Blood Pressure) monitor, embedding a sensor capable of measuring physiological data of a user (also called a patient), and wirelessly connected to a network, and a monitoring device, embedding a user interface and/or a data management flow, so that a care team may monitor the patient remotely.

The goal of RPM system is to continuously monitor a patient's vitals, such as his or her weight, blood pressure, blood oxygen saturation, etc.

### BACKGROUND

In RPM system, healthcare professionals remotely monitor patients using measuring devices. In the field of blood pressure, high BP data usually triggers a routine which requires the healthcare professionals (doctor or nurse) to reach out to the patient. However, some BP data are imperfectly taken and do not need immediate attention as they are external factors diminishing the criticality of the BP data, and some BP data are correctly high but not critical, as the criticality may depend on other factors.

Documents EP1726257, WO2017/089171, US20240172990A1, US2013267795, WO2019/200158, U.S. Pat. No. 8,438,038 disclose various solutions, some of them for RPM systems, some of them embedding user surveys or questions to enrich the measurement.

Withings™ provides body scales, blood pressure monitors (notably the BPM Pro 2™) and other devices suitable for RPM systems.

There is a need to provide improved RPM systems that will streamline the RPM process for professionals by decreasing the number of false positives, that are cases for which an intervention of the health professional is not required, and/or by better understand what's behind a BP data.

### SUMMARY

An aspect of the present disclosure is about a remote patient monitoring system (called “RPM system”), involving a user (or patient) and a professional (e.g., a physician, a nurse and any other professional) remotely taking care of the user thanks to connected physiological devices.

An aspect of the disclosure is a remote patient monitoring, RPM, system including a physiological measurement device comprising a physiological sensor, a user interface comprising a display and input hardware, comprising a wireless communication module, the RPM system further including a remote monitoring interface, configured to run on a remote monitoring device, wherein the remote monitoring device and the physiological measurement device are two distinct devices. The physiological measurement device is configured to send data to the remote monitoring interface using the wireless communication module. The physiological measurement device is configured to
obtain physiological data using the physiological sensor,
display on the display of the physiological measurement device a question with a plurality of predetermined responses, wherein the question and the plurality of predetermined responses form a plurality of contextual data
receive a predetermined response selected by the user using the input hardware of the BP monitor, wherein the question and the selected predetermined response form selected contextual data,
attribute a notification level to the physiological data using the physiological data and the selected contextual data, wherein the notification level is chosen amongst a plurality of available notification levels
display at the remote monitoring interface a display mode based on the attributed notification level.

Another aspect of the disclosure is a remote patient monitoring, RPM, system including:
a blood pressure, BP, monitor, comprising: a case, a user interface mounted on the case, with a display and input hardware, a blood pressure, BP, sensor configured to generate blood pressure data about the use, a wireless communication module,
a remote monitoring interface, configured to run on a remote monitoring device, wherein the remote monitoring device and the BP monitor are two distinct devices.

The BP monitor is configured to send data to the remote monitoring interface using the wireless communication module.

The RPM system is configured to:
obtain BP data using the BP sensor of the BP monitor,
display on the display of the BP monitor a question with a plurality of predetermined responses, wherein the question and the plurality of predetermined responses form a plurality of contextual data,
receive a predetermined response selected by the user using the input hardware of the BP monitor, wherein the question and the selected predetermined response form selected contextual data,
attribute a notification level to the BP data using the BP data and the selected contextual data, wherein the notification level is chosen amongst a plurality of available notification levels,
display at the remote monitoring interface a display mode based on the attributed notification level.

Thanks to these RPM systems, contextual data is used to change the notification level of the physiological measurement thereby helping the professional taking care of the patient to better address the actual critical physiological data. In the BP monitor case, having a fully-integrated device which takes the measurement, displays questions and receives the user input for the question, during a same measurement session, enables to actually receive user input regarding the BP measurement. The BP monitor is easy to use, even for people non familiar with it or elderly people.

The following paragraphs will present additional features of the RPM systems disclosed above.

In an embodiment, the plurality of available notification levels includes between 3 and 5 notification levels.

In an embodiment, the remote monitoring interface includes a plurality of display modes, wherein each display mode includes a dedicated layout corresponding to a notification level, such that the display modes display physiological data (e.g., BP data), for which contextual data are available, differently in function of the notification level. In particular, identical physiological data (e.g., BP data) with different selected contextual data which have led to the physiological data (e.g., BP data) having different notification levels have different display modes, and different display modes display the physiological data differently.

In an embodiment, the display mode includes a notification inbox, updated based on the attributed notification level.

In an embodiment, physiological data (e.g., BP data) is an average of several physiological data values (e.g., BP data value).

In an embodiment, displaying the question is performed in response to determining that the physiological data (e.g., BP data) verify a health condition. For example, the physiological data is under or above a threshold. The health condition may be editable (e.g., added, changed, updated, etc.) via the remote monitoring interface, such that the professional can customize the health condition to better suit his or her practice (specialty, disease, patients, etc.), in particular user by user.

In an embodiment, the display mode includes displaying the physiological data (e.g. BP data) along with the selected contextual data.

In an embodiment, the RPM further includes a server and the wireless communication module of the physiological device (e.g., the BP monitor) sends to the server the physiological data (BP data) and the selected contextual data and the remote monitoring interface receives from the server the physiological data (e.g., BP data), the selected contextual data and the attributed notification level.

In an embodiment, attributing the notification level includes querying a correspondence database using the physiological data (e.g., BP data) and the selected contextual data, wherein the correspondence database links a plurality of notification levels or of notification level variations to ranges values for the BP data and the contextual data. Notification level variations are to be applied to a preexisting notification level depending only on the physiological data (e.g., BP data)

In an embodiment, the correspondence database is stored on the server and the attribution is performed by the server.

In an embodiment, the correspondence database is stored on the BP monitor and the attribution is performed by the BP monitor.

In an embodiment, the RPM system further includes a remote monitoring device configured to run the remote monitoring interface, wherein the correspondence database is stored in the remote monitoring device.

In an embodiment, the correspondence database is editable via the remote monitoring interface, such that the professional can customize the correspondence database to better suit his or her practice (specialty, disease, patients, etc.), in particular user by user.

In an embodiment, the wireless communication module is a cellular module.

Another aspect of the disclosure is a remote patient monitoring method, using any of the RPM systems as presented above, the method comprising:
obtaining physiological data (e.g., BP data) using the physiological sensor (BP sensor of the physiological measurement device (e.g., BP monitor),
display on the display a question with a plurality of predetermined responses, wherein the question and the predetermined response form predetermined contextual data,
receive a predetermined response selected by the user using the input hardware, wherein the question and the selected predetermined response form selected contextual data,
attribute a notification level to the physiological data (e.g., BP data) using the physiological data (e.g., BP data) and the selected contextual data, wherein the notification level is chosen amongst a plurality of available notification levels,
display at the remote monitoring interface a display mode based on the attributed notification level.

Another aspect of the disclosure is a server or a method from the server point of view, for any of the RPM system as presented above:
the server sends to the physiological measurement device (e.g., BP monitor) a question database, the question database including at least one question and a plurality of predetermined responses for each question, wherein the question and the predetermined response form contextual data,
the server sends to the physiological measurement device (e.g., BP monitor) a health condition, wherein the health condition is to be checked with the physiological data (e.g., BP data),
the server receives from the physiological measurement device (e.g., BP monitor), physiological data (e.g., BP data) with a predetermined response of a question, wherein the question and the selected predetermined response form selected contextual data.
the server attributes a notification level to the physiological data (e.g., BP data), for example using a correspondence database as disclosed previously that is stored on the server,
finally the server sends out to the remote monitoring interface the physiological data (e.g., BP data), the selected contextual data along with the attributed notification level.

In an embodiment, there is provided a remote patient monitoring system, comprising:
a blood pressure (BP) monitor comprising:
   (a) a BP sensor configured to obtain BP data of a user;
   (b) a user interface comprising a display and input hardware;
   (c) a wireless communication module; and
   (d) a control unit configured to:
      (i) display on the display at least one question with a plurality of predetermined responses, the question and predetermined responses defining contextual data;
      (ii) receive, via the input hardware, a predetermined response selected by the user, thereby generating selected contextual data associated with the BP data; and
      (iii) transmit the BP data and the selected contextual data via the wireless communication module;
a server configured to receive the BP data and the selected contextual data, and to attribute a notification level to the BP data using a correspondence database linking notification levels or notification-level variations to ranges of BP values and contextual data; and
a remote monitoring interface executed on a remote monitoring device, the remote monitoring interface being configured to receive the BP data, the selected contextual data, and the attributed notification level from the server, and to display the BP data in a display mode selected based on the attributed notification level.

In yet another embodiment, there is provided a blood pressure (BP) monitor configured for use in a remote patient monitoring system, the BP monitor comprising:
a case;
a BP sensor configured to obtain BP data of a user;
a user interface mounted on the case, the user interface comprising a display and input hardware;
a wireless communication module; and
a control unit comprising at least one processor and a memory storing instructions which, when executed by the at least one processor, cause the BP monitor to:
obtain BP data using the BP sensor;
display, on the display, at least one question together with a plurality of predetermined responses, the question and the plurality of predetermined responses defining contextual data;
receive, via the input hardware, a predetermined response selected by the user, thereby generating selected contextual data associated with the BP data;
form enriched BP data including the BP data and the selected contextual data; and
transmit, via the wireless communication module, at least the enriched BP data toward a remote monitoring interface executed on a remote monitoring device, the enriched BP data being usable by the remote monitoring interface to determine a notification level and a display mode for presentation to a healthcare professional.

In another embodiment, there is provided a non-transitory computer-readable medium storing instructions that, when executed by at least one processor of a blood pressure (BP) monitor, cause the BP monitor to perform a method comprising:
obtaining BP data using a BP sensor of the BP monitor;
displaying, on a display of the BP monitor, at least one question together with a plurality of predetermined responses, the question and predetermined responses defining contextual data;
receiving, via input hardware of the BP monitor, a predetermined response selected by a user, thereby generating selected contextual data associated with the BP data;
forming enriched BP data including the BP data and the selected contextual data; and
transmitting, using a wireless communication module of the BP monitor, the enriched BP data toward a server or a remote monitoring interface configured to attribute a notification level to the BP data based on the enriched BP data and to display the BP data in a display mode corresponding to the notification level.

The additional features disclosed previously also applicable to the server, when relevant.

### DESCRIPTION OF THE DRAWINGS

Other features will be visible on the following non limitative drawings:

FIG. 1 shows a remote patient monitoring, RPM, system, according to an embodiment of the disclosure,

FIG. 2 shows a BP monitor according to an embodiment of the disclosure,

FIG. 3 FIG. 2 shows a remote monitoring interface run on a monitoring device according to an embodiment of the disclosure,

FIG. 4 shows a server according to an embodiment of the disclosure,

FIG. 5 shows BP data obtained and displayed by the BP monitor according to an embodiment of the disclosure,

FIG. 6 shows examples of questions displayed by the BP monitor according to an embodiment of the disclosure,

FIG. 7 shows an example of a display mode with notification levels on the remote monitoring interface according to an embodiment of the disclosure,

FIG. 8 shows an example of a display mode with enriched BP data on the remote monitoring interface according to an embodiment of the disclosure,

FIG. 9 shows an correspondence database according to an embodiment of the disclosure,

FIG. 10 shows a RPM method performed by a RPM system, according to an embodiment of the disclosure,

FIG. 11 shows a RPM method performed by the server, according to an embodiment of the disclosure.

### DETAILED DESCRIPTION

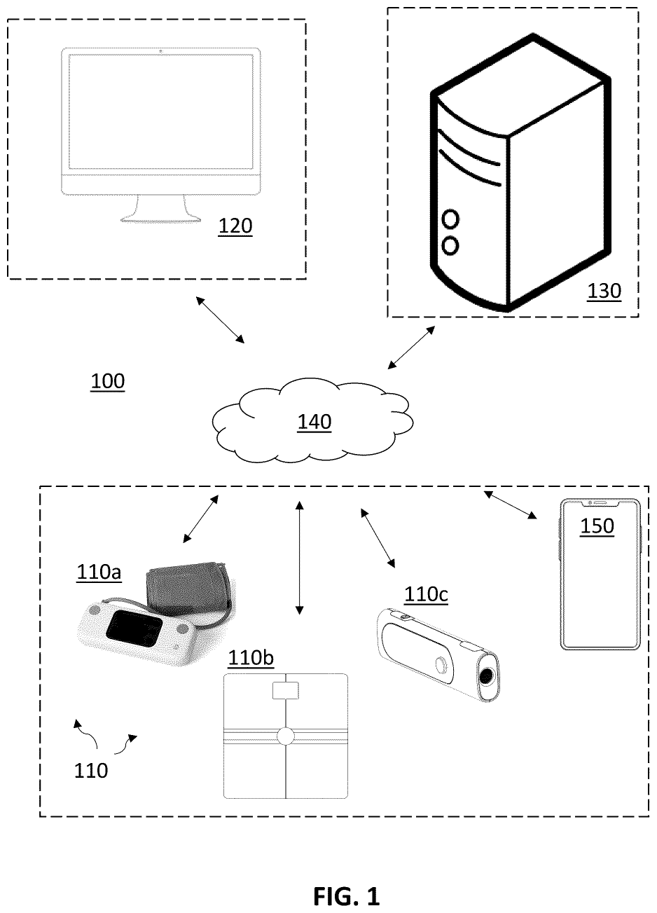

FIG. 1 illustrates an exemplary embodiment of a remote patient monitoring system 100, called RPM system 100. As used herein, the term “remote” in the expression “remote patient monitoring system” refers to the fact that the professional and the patient operate the respective components of the RPM system 100 from different physical locations, such that the professional does not need to be co-located with the patient or with the physiological measurement device during acquisition of physiological data. The term “remote” encompasses situations in which the professional is located in a separate room, building, facility, neighborhood, city, region, or any geographically distinct environment relative to the patient. It further encompasses implementations in which data is transmitted across local-area networks, wide-area networks, cellular networks, or the Internet, and does not require real-time synchronous communication between the patient and the professional. The term therefore excludes arrangements in which the professional directly observes or controls the measurement device at the time of measurement, and instead defines a technical architecture in which the physiological measurement device 110, the server 130, and the remote monitoring interface 120 operate cooperatively to allow the professional to review, interpret and act upon physiological data obtained at a distance.

The embodiment depicted in FIG. 1 is presented in simplified, schematic form for purposes of illustration and is not intended to be limiting with respect to the number, type, physical arrangement or implementation of the components shown, which may vary between different deployments of the RPM system 100. The RPM system includes at least one physiological measurement device 110 (here a blood pressure monitor 110a, called BP monitor, and/or a body scale 110b, and/or a multiscope 110c (such as disclosed in EP24217550 or devices called BeamO™), but could comprise other devices, such as those disclosed in EP4386383 to Withings™, or devices U-Scan™ developed by Withings™), a remote monitoring interface 120, a server 130 and a communication network 140. In general, each physiological measurement device 110 may comprise one or more physiological sensors, a local user interface, a power supply (for example a battery or mains power adapter), and a communication module enabling the device 110 to exchange data with the communication network 140 either directly or via an intermediate device, and a plurality of distinct physiological measurement devices 110 may be associated with a single user account within the RPM system 100. The RPM system 100 may comprise also a mobile device 150, such as a smartphone or smartwatch. In various embodiments, the mobile device 150 is configured to execute a patient-facing software application that implements a close monitoring interface for the user, to act as a gateway relaying data between the physiological measurement device 110 and the server 130, to locally store historical physiological measurements and contextual data, and/or to receive configuration information, alerts and software or firmware updates from the server 130 and forward them to the physiological measurement device 110. The mobile device 150 may, by way of non-limiting example, be implemented as a smartphone, a smartwatch, a tablet computer, a laptop computer or any other portable electronic device including a processor, a memory and a wired and/or wireless communication interface. The remote monitoring interface 120 is configured to run on a remote monitoring device (not shown on FIG. 1), which is remotely arranged from the BP monitor 110a. The remote monitoring interface 120 may be implemented as software instructions executed by the remote monitoring device and may take the form of a native application, a web application, a web portal or another graphical user interface configured to receive, organize, filter, display and allow interaction with physiological data and contextual data transmitted from one or more physiological measurement devices 110. The remote monitoring device may, for example, be a desktop computer, a laptop computer, a tablet computer or another professional-grade computing device used within a clinic, hospital or telemedicine facility. The mobile device 150 may be configured to run a close monitoring interface, suitable for the user. The communication network 140 schematically represents any suitable wired and/or wireless communication infrastructure coupling the physiological measurement device 110, the remote monitoring device and the server 130, and may include, without limitation, one or more local-area networks (LANs), wide area networks (WANs), cellular networks, the Internet, and/or short range wireless links-such as Wi Fi® or Bluetooth®, arranged in any appropriate topology.

The RPM system 100 is used by various people, who are defined as follows: the user of the BP monitor 110a is referred to as a patient and the user of the monitoring device 140 is referred to as a professional, since he or she is a healthcare professional, such as a nurse or a physician. For clarity, in the context of the present description the terms “patient” and “user” may be used interchangeably to designate the individual whose physiological data are being measured and monitored, regardless of whether that individual is acutely ill, chronically ill or simply under preventive or routine follow-up care, and the term “professional” is intended broadly to encompass any authorized healthcare provider or member of a care team who is permitted to consult and/or act upon the monitored data, such as physicians, nurses, nurse practitioners, physician assistants, pharmacists, medical technicians and other allied health professionals. The professional and the user in a RPM system are usually not in the same room, but usually at their respective work or living locations, except for a specific appointment, such as a follow checkup. In typical home-monitoring scenarios, the patient operates the BP monitor 110a from a residence, workplace or assisted-living facility, while the professional accesses the remote monitoring interface 120 from a clinic, hospital, telemedicine center or other professional environment, so that blood-pressure measurements and associated contextual data can be obtained by the patient at one time and location and reviewed or acted upon by the professional at a different time and/or location. Although FIG. 1 is described, for ease of explanation, with reference to a single patient and a single professional, the RPM system 100 can be configured such that a given professional monitors physiological data associated with a plurality of user accounts, each corresponding to a distinct patient enrolled in a remote patient-monitoring program. In some embodiments, the RPM system 100 may also be accessed in a limited and controlled manner by additional users, such as technical administrators responsible for maintaining the system or configuring user accounts, while preserving strict segregation between technical administration functions and medical decision-making functions performed by the professional.

In a typical situation, the BP monitor 110a measures blood pressure data (BP data) on the patient during a measurement session initiated by the user or automatically scheduled by the RPM system 100 and sends the BP data to the server 130 via the communication network 140, optionally after temporarily storing the BP data and associated metadata (such as a timestamp, a user account identifier, and/or an identifier of the BP monitor 110a) in an internal memory of the BP monitor 110a. The BP data may be transmitted in raw form (for example, systolic and diastolic pressure values and, where available, heart-rate values) and/or in processed form (for example, averaged values over several inflations, quality indicators or flags indicative of artefacts), using any suitable wired and/or wireless communication protocol supported by the communication network 140 and, in some embodiments, in an encrypted or otherwise secured format suitable for medical data. Then the server 130 sends the BP data or a data obtained from the BP data to the remote monitoring device 120, via the communication network 140, for example immediately after reception of new BP data, periodically in batch mode, or in response to a request initiated from the remote monitoring interface 120. The “data obtained from the BP data” may include, without limitation, filtered or validated BP values, aggregated statistics over a plurality of measurements, indicators comparing the BP data with reference ranges or individualized thresholds defined for the patient, trend information over time, and/or a notification level attributed to the BP data as described herein. In certain embodiments, the server 130 also associates the BP data with other physiological measurements or administrative data belonging to the same user account, thereby enabling the professional to access a consolidated and contextualized view of the patient's status through the remote monitoring interface 120. As it will be disclosed in more details, the RPM system 100 of the present disclosure also allows adding contextual information pertaining to the BP data. Such contextual information may be collected on the BP monitor 110a itself, on the mobile device 150, and/or on another physiological measurement device 110, and may comprise, for example, responses to one or more questions displayed to the patient, information about recent activity or rest, medication-intake information, lifestyle data or other circumstances that may influence the interpretation of the BP data. The contextual information is stored in association with the corresponding BP data in the RPM system 100 so that both the user and the professional can interpret a given BP measurement in light of the circumstances under which it was obtained. The close monitoring interface of the mobile device 150 may allow the user to see the BP data or any related-data, including, for example, the contextual information linked to each BP measurement, summaries or graphs showing the evolution of BP data over time, and explanatory or feedback messages generated by the RPM system 100 on the basis of the BP data and/or an attributed notification level, thereby improving the user's understanding of his or her condition while the professional remotely reviews the same enriched information.

The RPM system 100 is aimed at gathering blood pressure data on the patient, via the BP monitor 110a, and at providing them to the professional, via the monitoring device 120. More specifically, the RPM system 100 is designed to establish a continuous or semi-continuous flow of clinically relevant information from the patient's environment to the professional's remote monitoring interface, thereby enabling timely detection of abnormal physiological trends and facilitating efficient triage of patients under remote care. The BP monitor 110a serves as the primary physiological measurement device for obtaining BP data, which may include systolic and diastolic values, heart-rate measurements, quality indicators and any associated contextual data generated during the same measurement session. Once obtained, these data are transmitted over the communication network 140 to the server 130, where they may be stored, processed, enriched with metadata, and linked to a corresponding user account. The remote monitoring interface 120 then retrieves the BP data—optionally in enriched form or accompanied by an attributed notification level—and presents them to the professional in a structured and clinically actionable display mode. In this manner, the RPM system 100 not only transfers raw BP measurements but also provides the professional with enhanced situational awareness regarding the circumstances under which each measurement was taken, ultimately improving the quality and efficiency of remote therapeutic decision-making.

The BP monitor 110a, the remote monitoring interface 120 and the server 130 all exchange data using the communication network 140, which may be a hybrid network (ethernet, Wi-Fi, 3G, 4G, 5G, etc.).

### The BP Monitor 110a

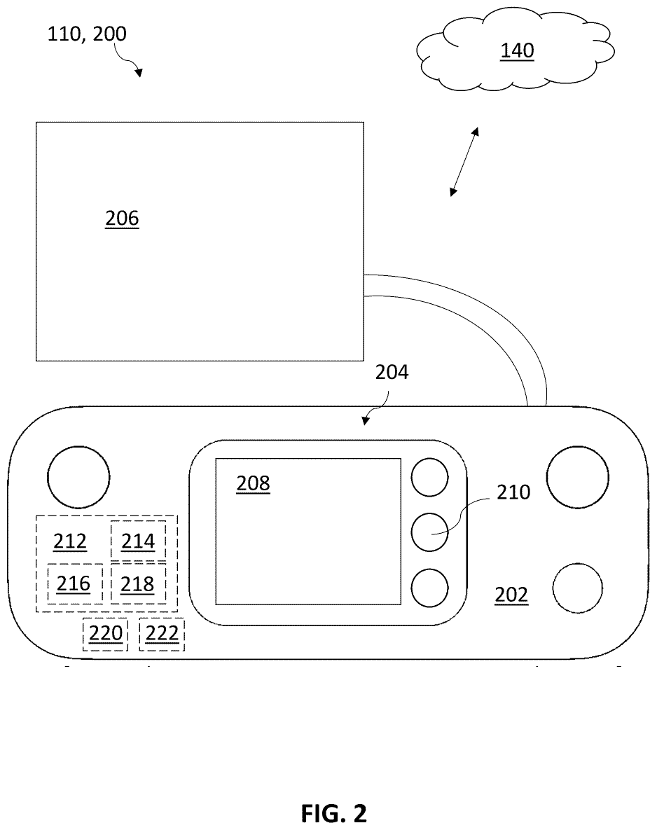

FIG. 2 illustrates a BP monitor 200 corresponding to the BP monitor 110a of FIG. 1. The BP monitor 200 includes a case 202, a user interface 204 mounted on the case 202 and a blood pressure sensor 206. The user interface 204 includes a display 208 and an input hardware 210. The display 208 may be a screen, as visible on FIG. 2, or a speaker. From the BP monitor 200 standpoint, the display 208 is aimed at providing information to the user while the input hardware 210 is aimed at receiving information from the user. The input hardware 210 may comprise buttons, as illustrated. As the BP monitor 200 is targeting elder people, the simpler the input hardware 210 the better. Alternatively or complementarily, the input hardware 210 may comprise a tactile screen.

The blood pressure sensor 206 is shown as a cuff on FIG. 2 but the blood pressure sensor 206 typically includes the cuff, a pump (connected to the cuff via a flexible pipe) and a pressure sensor configured to generate pressure data in the cuff.

As schematically shown on FIG. 2, the BP monitor 200 includes a control unit 212, comprising a processor 214, a memory 216 and a I/O (in/out) interface 218. The control unit 212 controls the overall operation of the BP monitor 200, including measurement acquisition, user interaction, and data communication. The memory 216, which includes storage, is configured to store instructions, which, when executed by the process, allows the BP monitor 200 to notably take BP measurement, display information on the screen, receive user input. The I/O interface 218 manages the interactions of the control unit 212 with the other components. A communication module 220 enables the BP monitor 200 to exchange data, typically in a wireless manner with the communications network 140. A battery 222 stores energy to allow the BP monitor 200 to work without being electrically plugged, thereby enabling routine home-based use and ensuring that measurement and contextual data can be obtained even in the absence of a wired power source. In some embodiments, the battery 222 is rechargeable and cooperates with a power-management subsystem that monitors battery status and optimizes energy consumption during measurement and wireless transmission.

In an embodiment the communication module 220 is a cellular module capable of exchanging data on a cellular network of the communication network, so that no mobile device 150 is required to forward any data generated by the BP monitor 110a.

The BP monitor 110a may be shared by several users. To that end, each user has a user account. Data generated for a same user is associated with the same user account.

### The Remote Monitoring Interface 120

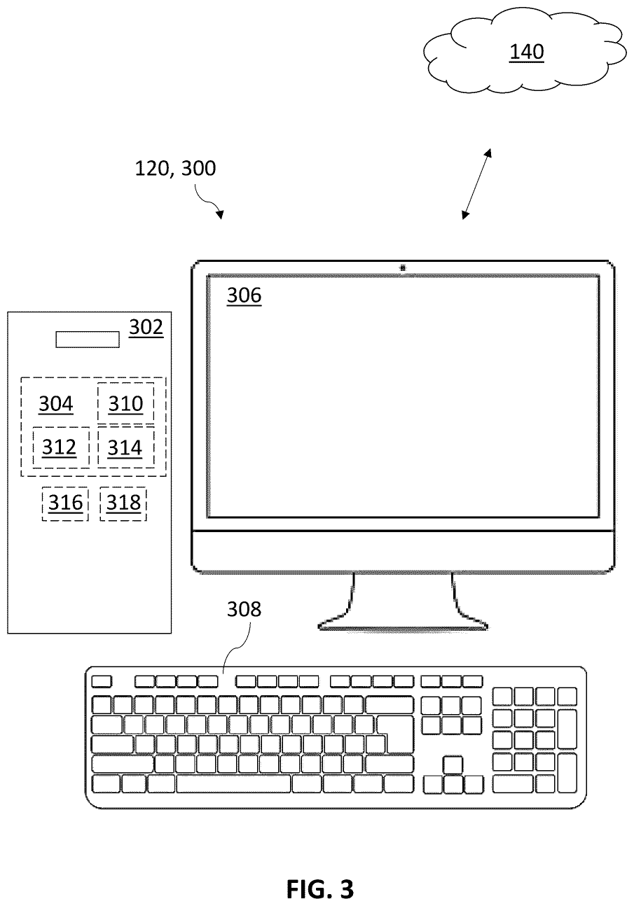

FIG. 3 illustrates a remote monitoring interface 300 corresponding to the remote monitoring interface 120 of FIG. 1. The remote monitoring device interface 300 can be run by a monitoring device 302 using a control unit 304 capable of processing data, including receiving data from the server 130 and displaying data. The remote monitoring interface 300 is configured for permitting an interaction with the professional. To that end the remote monitoring interface 300 may include a display 306, such as a screen, and a hardware input 308, such as a keyboard and/or a mouse, or a trackpad. The remote monitoring device 302 is typically a personal computer (e.g., a laptop).

The remote monitoring interface 300 is typically a software-based interface, which can be deployed as an app, for example accessible on an Appstore, or a webapp or a web page accessible from a navigator. The remote monitoring interface 300 is generally provided as a SaaS (software as a service).

As schematically shown on FIG. 3, the control unit 304 comprises a processor 310, a memory 312 and a I/O (in/out) interface 314. The memory 312, which includes storage, is configured to store instructions, which, when executed by the process, allows the monitoring device 302 to notably display information on the display 306 and receive user input via the hardware input 308. The I/O interface 314 manages the interactions of the control unit 302 with the other components. A communication module 316 enables the monitoring device 302 to exchange data with the communication network 140. A battery 318 may store energy to allow the monitoring device to work without being electrically plugged.

To qualify as a RPM system 100, the remote monitoring interface 120 is generally not activated on the BP monitor 110a or the mobile phone of the user, but only on the monitoring device 302 of the professional. In particular, the remote monitoring interface 120 is intentionally restricted to devices operated by authorized healthcare professionals so as to preserve a clear functional distinction between patient-facing components of the RPM system and professional-facing components. By contrast, the BP monitor 110a and any mobile device 150 associated with the user typically execute only patient-oriented interfaces, such as simplified measurement displays or close-monitoring interfaces, and do not provide access to the advanced monitoring dashboard, analytics tools, or multi-patient management capabilities available through the professional's remote monitoring device 302.

The remote monitoring interface 120, 300 gives access to the professional to data from various user accounts, notably (and only) the user accounts of the user which are under a RPM program by the professional.

### The Server 130

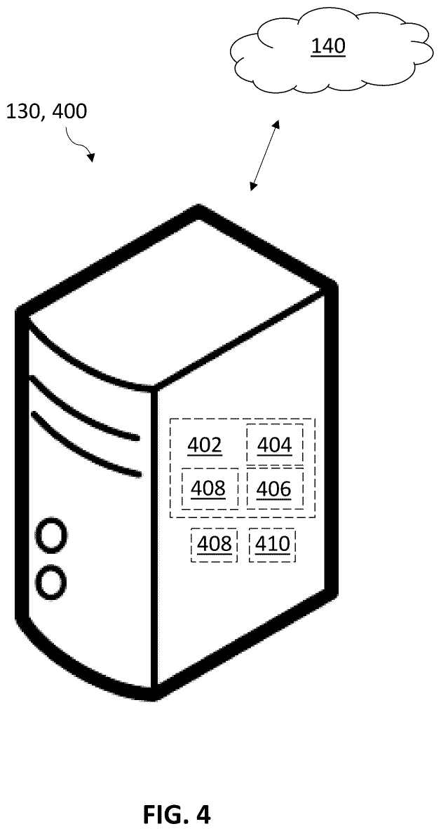

FIG. 4 illustrates a server 400 corresponding to the server 130 of FIG. 1. The server 130 is a remote hardware unit configured to receive, store and process data received from the BP monitor 110a and to send the BP data or any related data to the remote monitoring interface 120. Conversely, the server 130 is also configured to receive, store and process data received from the remote monitoring interface 120 and to send the data or any related data. The server 130 is typically remotely located from the BP monitor 110a and the remote monitoring interface 120. The server 130 may be located in a datacenter, for example a datacenter provided by a datacenter provider or by the BP monitor provided or by the hospital data center of the professional.

The server 400 may store the user accounts (with all the associated data) in a centralized manner and acts as a cloud backup.

As schematically shown on FIG. 4, the server 400 includes a control unit 402, comprising a processor 404, a memory 406 and a I/O (in/out) interface 408. The memory 406, which includes storage, is configured to store instructions, which, when executed by the process, allows the server 400 to query a correspondence database which will be disclosed in more detail later on. The I/O interface 408 manages the interactions of the control unit 402 with the other components. A communication module 408 enables the BP monitor 200 to exchange data, typically in a wired manner with the communications network 140.

The server 130, 400 may include a plurality of servers, for example a first server provided by the BP monitor provider and a second server provided by the professional's facilities, the first and the second server exchanging data via API (application programming interface), or a server storing a user EHR (electronic health records). From the RPM system of the present invention, any combination of those servers is considered as the server.

### The RPM System

The description will now explain in more detail how the RPM system 100 works. In particular, the RPM system 100 is aimed at providing contextual information to the BP data so that the RPM system may adapt the display of the BP data at the remote monitoring interface 120. As used herein, “contextual information” or “contextual data” refers to any data element, qualitative or quantitative, that describes the circumstances, conditions, behaviors, or external factors surrounding the acquisition of a given BP measurement. Contextual data may include, for example, responses to predefined questions presented to the user (e.g., whether the user has taken antihypertensive medication, whether the user has rested for a prescribed period prior to the measurement, whether the user recently exercised, slept poorly, consumed a salty meal, or experienced stress). Contextual data may further include other environmental, behavioral, physiological or temporal indicators that influence or explain the BP data, such as the time of day, adherence to medical instructions, recent symptoms, or other related lifestyle inputs. In the RPM system 100, such contextual data is deliberately captured during or immediately after the measurement session, and is associated with the corresponding BP data to form enriched BP data that more accurately represents the user's health status. This provides helpful assistance to the professional in sorting the users that need immediate support because of their BP data from users whose BP data have been negatively influenced by their behavior (lack of medication, physical effort before the measurement, etc.). Accordingly, the use of contextual data enables the professional to distinguish clinically critical BP readings from readings that may be transiently elevated or otherwise distorted by modifiable factors, thereby improving triage efficiency and reducing unnecessary interventions.

All data of a same user is associated with a user account.

### BP Data

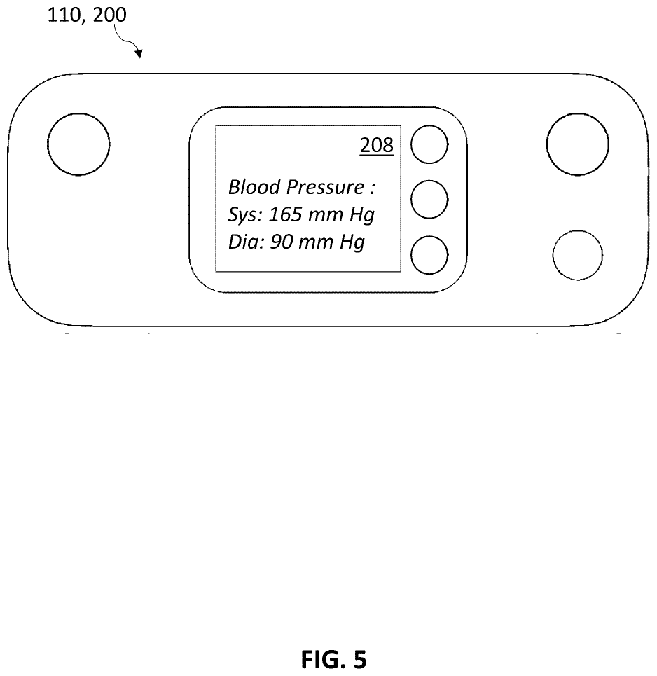

The BP monitor 110a is configured to obtain BP data from the user. This is well known and will not be disclosed in more detail. The BP data may be displayed on the display 208, as shown on FIG. 5. However, as the BP data may be influenced by external factors, as disclosed previously, the RPM system requests contextual information.

In an embodiment, the BP data is an average value of several measurements, either taken successively in the same session (for example three successive BP measurements with a one-minute rest between), or taken on different days.

### Questions

To gather contextual information, the BP monitor 110a is configured to display, on the display 208, one or more questions for the user. Typically, the question is displayed after the BP data has been displayed.

In an embodiment, the question is displayed regardless of the value of the BP data; that is to say the BP data is not checked. This means that contextual information will be generated for every BP data, and thus for each BP measurement taken by the user. This allows the professional to gather much more intelligence about the user's health but this makes the BP measurement process more cumbersome for the user, who might lose patient and stop taking BP measurement.

In another embodiment, the question is displayed if the BP data verifies a health condition. This avoids generating contextual information for BP data that are considered for example as healthy. The health condition may be a BP data range value, for example a BP systolic pressure value or range. The health condition may be that under 119 mm Hg for the BP systolic pressure, no question is displayed by the BP monitor 110a.

The health condition may be tailored for each user, notably by the professional, using the remote monitoring interface 120.

The health condition enables to avoid interacting with the user when it is not necessary and to avoid generating contextual data for the professional when it is not necessary either. In particular, the “health condition” acts as a configurable decision rule or threshold—defined, for example, by one or more BP value ranges, combination criteria involving systolic or diastolic pressure, or additional physiological or contextual parameters—that determines whether the RPM system 100 should trigger the display of a question and thereby initiate the creation of contextual data. By selectively prompting the user only when the measured BP data meet or exceed such a health condition, the RPM system 100 prevents the accumulation of redundant, low-value contextual information and focuses the user interaction on those situations in which contextual data may materially affect the interpretation of the BP measurement. The goal of the RPM system of the present disclosure is to streamline the process to improve the quality of the remote monitoring by the professional. This streamlining benefits both users and professionals: the user experiences a simpler, less burdensome measurement workflow, while the professional receives fewer non-actionable data points and a cleaner dataset centered on clinically meaningful events. Therefore any “noise” has to be avoided. As used herein, “noise” refers to any data, prompt, or interaction that does not contribute to improved clinical understanding or decision-making, including unnecessary user queries, contextual data associated with normal BP readings, or repeated information that does not change the notification level attributed to the measurement. Reducing such noise enhances the signal-to-noise ratio of the patient's data profile, thereby enabling the professional to more rapidly identify true outliers, trends of concern, or patterns requiring medical follow-up.

To each question corresponds a limited number of responses that the user can select on the display using the input hardware, called predetermined responses.

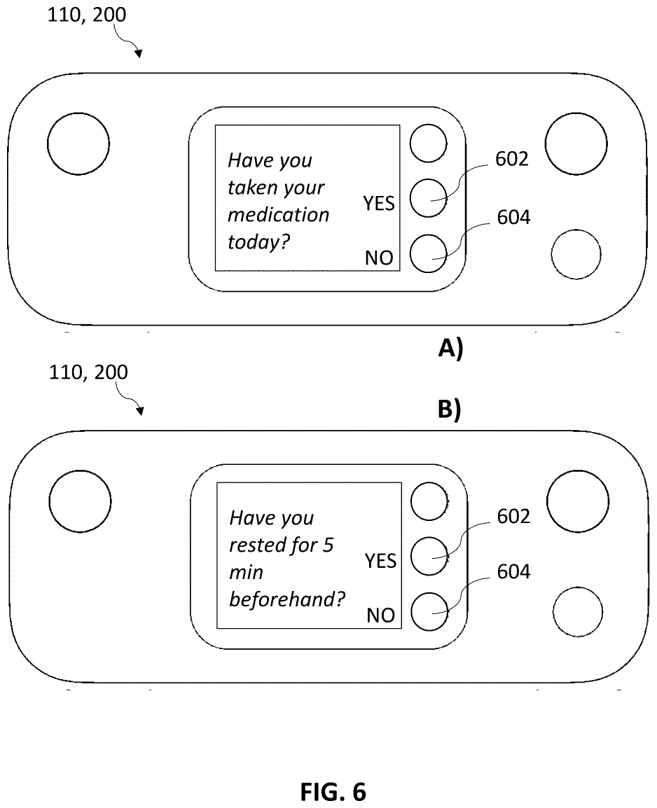

In an embodiment, the question is a yes/no question and one button is attributed to YES and another button is attributed to NO. This makes the BP monitor 110a particularly simple to use, notably for elder patients. Examples A) and B) on FIG. 6 show yes/no question on the BP monitor 110a, with buttons 602, 604 attributed to respectively YES and NO.

In an embodiment, the question is a series of successive sub-questions and the possible responses for each question is a number, so that the predetermined responses for the question (here the series of successive sub-questions) is also number, for example all the possible value for sum of all the numbers answered to the series of successive question or all the possible value of average thereof.

The questions and the predetermined responses for each question are stored in a question database. Each question with the plurality of predetermined responses form contextual data that can be selected by the user using the hardware input 210. The question and the selected predetermined question form selected contextual data.

The following list provides non-limiting examples of questions with associated responses:

have you taken your medication today? Yes/No; have you rested for 5 min before taking your BP? Yes/No; did you sleep well? Yes/No; have you eaten a particularly salty meal? Yes/No. Other examples of questions are provided in the documents cited in the introduction.

The question database is stored at the BP monitor 110a, while a master question database is stored at the server 130 and a fraction of it (the question database thus) is downloaded from time to time by the BP monitor 110a. The master question database functions as a centralized repository of predefined questions and associated predetermined responses, organized for use across multiple users, care programs, or clinical pathways. This master database is structured to include both general-purpose questions—applicable to a wide range of patients—and specialized questions tailored to particular conditions, treatment regimens, comorbidities, or monitoring protocols. The master question database may be updated, notably through the remote monitoring interface 120, by the professional, for example to draft specific questions for certain users. The master question database and the question database may be identical. Such updates may include adding new questions, modifying existing ones, adjusting response options, or activating or deactivating entire question sets. In some embodiments, updates may also be generated automatically based on clinical guidelines, institutional protocols, or evidence-based rules implemented on the server 130, thereby ensuring that the master question database remains aligned with evolving medical standards. The master question database and the question database may be identical. In this configuration, the BP monitor 110a stores the complete set of available questions, thereby enabling full flexibility in contextual data capture without relying on selective downloads. However, should there be several health issues to be addressed with different needs as for contextual data, the master question database will be populated with many more questions. In such cases, the BP monitor 110a downloads only a relevant subset of the master question database—selected according to user-specific criteria such as diagnosed conditions, risk category, physician-defined monitoring plans, or the parameters of a particular RPM program. The subset selection may be performed by the server 130, which associates question sets with the user's account profile, and periodically transmits updates to the BP monitor 110a. As a result, the BP monitor 110a maintains a streamlined, context-appropriate question database containing only those questions deemed useful for interpreting that user's BP data, thereby minimizing device complexity while maximizing clinical relevance.

The measured BP data and the selected contextual data are associated together and are sent to the server 130.

### The Notification Level and the Display Modes

One of the issues of the notification levels is to manage the professional's attention span and the professional's time to address users in need. However, some BP data may be abnormally high but may be less critical than others, notably because the user's recent behavior has tended to increase the BP data value. For example a plurality of notification levels is available for managing the BP data at the remote monitoring interface 120.

For example, there may be three levels of notifications levels: green, yellow and red. A green level means that the professional does not need to be alerted, while the yellow level means that the professional should have a look at the BP data and, finally, the red level means that the professional needs to look at the BP data as soon as possible. A fourth level of notification may be implemented, for cases that are not urgent but need to be looked at: for example, a BP data for a patient suffering from hypertension that is in the normal BP value ranges.

Based on the notification level, the remote monitoring interface 120 may display various display modes. Each display mode corresponds to a predetermined visual presentation, layout, or emphasis scheme that conveys to the professional the urgency, relevance, or clinical significance of the BP data to which the notification level has been attributed. The display modes may differ, for example, in terms of color coding, iconography, highlighting, grouping within an alert inbox, sorting priority, font size, or additional metadata made visible to the professional. The various display modes are to be assessed for BP data with contextual data, meaning that identical BP data with different selected contextual data which have led to the BP data having different notification levels have different display modes. Accordingly, the RPM system 100 ensures that the display output is not solely a function of the numerical BP value, but is dynamically adapted to incorporate the contextual factors that influence clinical interpretation, such that otherwise identical measurements may be rendered with markedly different urgency indicators. Said otherwise, different display modes display the BP data differently. In some embodiments, the display modes additionally determine whether enriched BP data (i.e., BP data associated with contextual data) is shown in expanded or collapsed form, whether explanatory messages are provided, or whether the measurement is escalated within the professional's workflow. This adaptive display paradigm improves the readability and triage efficiency of the remote monitoring interface 120, enabling the professional to quickly distinguish between clinically meaningful events and measurements of lower concern.

In an embodiment, the layout between various display modes varies upon the notification level. There is a predetermined number of display modes, corresponding to the number of notification levels for example.

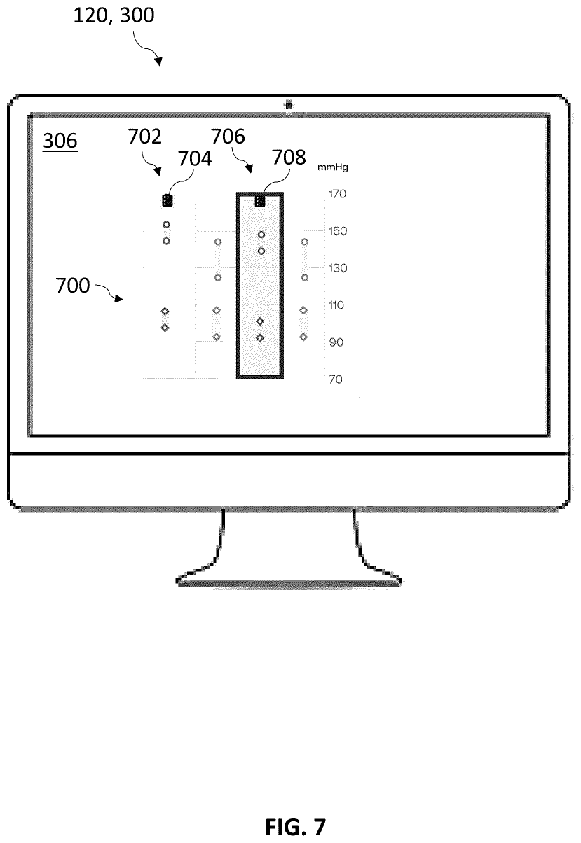

For example, BP data with a red notification level appear with a flag, while BP data with a green notification level does not stand out. In certain embodiments, a red notification level may be represented by a prominent visual indicator such as a red banner, a flashing icon, a bold outline, or a dedicated alert symbol designed to immediately draw the professional's attention. Conversely, a green notification level may be rendered using a neutral or understated display mode that intentionally blends into the background of the remote monitoring interface 120, thereby signaling that no urgent clinical action is required. FIG. 7 illustrates an example of display modes 800, in which measurement 702 and measurement 706, both high BP data with contextual data, do not appear in the same way because measurement 702 includes contextual data (see icon 704) about the BP data that triggered a lower notification level than measurement 706 which includes contextual data (see icon 708) about the BP data that triggered a higher notification level. In the illustrated embodiment, icon 704 may correspond to contextual data suggesting that the elevated BP value is partially attributable to a reversible or non-critical factor—such as insufficient rest or a missed medication dose—thereby reducing the severity attributed to measurement 702. Icon 708, by contrast, may correspond to contextual data suggesting a more concerning clinical picture, such as proper rest and medication adherence despite persistently high BP values, thereby increasing the severity attributed to measurement 706. A higher notification level implies a higher emergency character than a lower notification level. Accordingly, the RPM system 100 visually differentiates the two measurements in a manner proportionate to their respective urgency levels. Measurement 706 has for example a colored background while measurement 702 does not have one. Other visual distinctions may also be employed, including the use of different font weights, color gradients, border styles, icons, or placement within a notification inbox. Just by looking at the display modes, the professional is informed that measurement 706 presents a higher emergency character than measurement 702. This enables rapid triage, reduces cognitive load on the professional, and supports efficient allocation of clinical attention within a multi-patient monitoring environment.

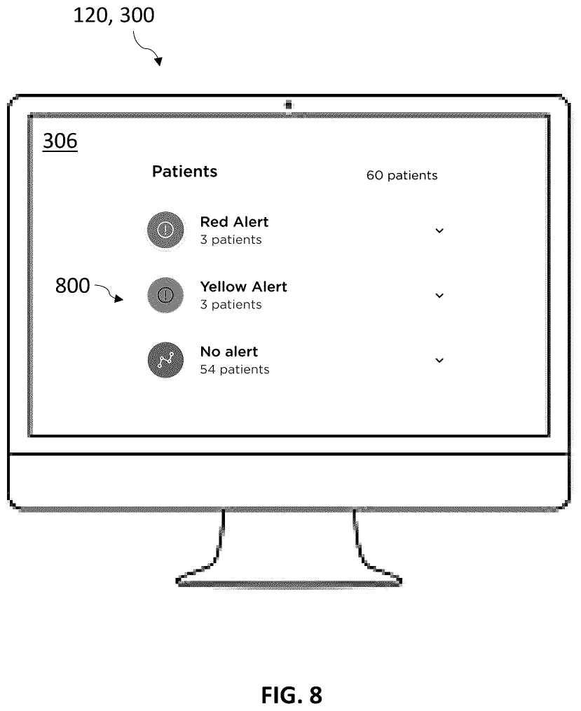

In an embodiment, the layout between various display modes is standard for all notification levels but there is a notification inbox, which is updated based on the attributed notification level. FIG. 8 illustrates an example of a notification inbox 800, in which the green level is called “no alert”, yellow level is called “yellow alert” and red level is called “red alert”.

In an embodiment, the remote monitoring interface 120 displays the enriched BP data, that is to say that the selected contextual data are displayed along with the BP data. The RPM system 100 may slightly reformulate the selected contextual data to render it clearer. For example, the contextual data “have you taken your medication today? No” may be turned into “medication not taken today”.

The notification level may also take into account repetition of the same contextual data over time. For example, the notification level for BP data of a user who has not taken his or her medication two days in a row might be increased by one level each additional day.

### The Correspondence Database

In particular, in the present disclosure, the RPM system 100 attributes a notification level to the BP data using these BP data and the selected contextual data. The selected contextual data are aimed at increasing or decreasing the notification level of the BP data compared to a non-contextual notification level, which is based only on the BP data. Selected contextual data associated with an increase of BP data value (e.g., no rest before measurement or no hypotensive medication taken) tend to decrease the notification level (since external factors temporally increase the BP data value, the high BP data value is less critical). Selected contextual data associated with an accurate assessment or a decrease of BP value tend to increase the notification level (since the BP data value is at its true value or lower, a high BP value is more critical), for a same BP value.

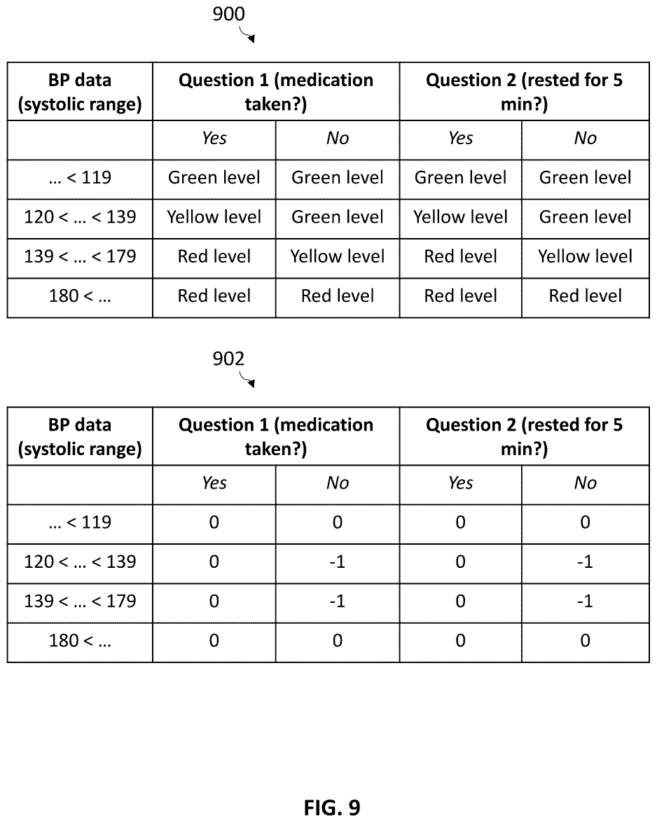

A way to determine the notification level may involve a correspondence database 900, 902 shown on FIG. 9.

In an embodiment, the correspondence database 900 provides a notification level to the BP data with the selected contextual data. The correspondence database 900 functions as a structured decision resource—implemented, for example, as a lookup table, rule set, decision tree, algorithmic model, or combination thereof—configured to evaluate both physiological measurements and contextual information in a unified manner. To that end, the correspondence database 900 takes, as inputs, the BP data (their value, for example the diastolic and systolic pressure values) and the question with the selected contextual data, and returns, as output, a notification level for the inputted BP data. More specifically, the correspondence database 900 receives (i) one or more BP parameters, such as systolic pressure, diastolic pressure, pulse rate, measurement quality indicators, or BP value ranges, and (ii) the contextual data associated with that BP data, including the specific question presented and the corresponding predetermined response selected by the user. The correspondence database 900 then identifies the matching entry or rule that corresponds to the particular combination of BP values and contextual data, and maps that combination to one of a plurality of available notification levels (e.g., green, yellow, red, or intermediate categories). In doing so, the database effectively integrates both clinical severity and situational relevance, ensuring that the resulting notification level reflects not only the magnitude of the BP values but also the circumstances under which they were obtained.

In this embodiment, the correspondence database 900 therefore includes, for at least the question displayed on the BP monitor 110a (in an embodiment, for all the question database stored in the BP monitor 110a; in another embodiment, for all the questions of the master question database), a table giving a notification level for all the combinations of BP values (e.g., ranges of BP data values) and predetermined responses for all the questions of the database.

In an embodiment, the correspondence database 902 provides a notification level variation to the BP data with the selected contextual data. To that end, the correspondence database 900 takes, as inputs, the BP data (their value, for example the diastolic and systolic pressure values) and the question with the selected contextual data, and returns, as output, a notification level variation for the inputted BP data. The variation is then to be applied to a pre-existing notification level for BP data only (without contextual data).

The correspondence database 900 may include, as entries:
ranges for the BP data value. For example, the ranges for BP data systolic values may be: under 119 mm Hg, between 120 and 139 mm Hg, between 139 and 179 mm Hg, and above 180 mm Hg (this is only an illustrative example with no medical value whatsoever),
the predetermined responses of each question, such as “have you taken you medication today?” Or “have you rested for 5 minutes before taking the measurement?”). At each intersection of the entries, a notification level is associated or a notification level variation is associated. The choice of notification level or notification level variation may be defined by the professional, for example through the remote monitoring interface 120. This determination may be carried out by the professional user by user, and/or disease by disease, to allow of an improved customization of the notification level. Similarly, the number of notification levels may be determined by the professional, depending on his or her own practices or on the user's needs.

For example, assuming there are only two ranges and two questions with two predetermined responses (such as YES or NO questions), then the correspondence database includes 2 ranges×2 questions×2 predetermined responses=8 cells.

For example, as in correspondence database 900 of FIG. 9, assuming there are only four ranges and two questions with two predetermined responses (such as YES or NO questions), then the correspondence database 900 includes 4 ranges×2 questions×2 predetermined responses=16 cells.

In practice, the correspondence database 900 may then be queried to return a notification level. The query includes inputting the BP data value (such as the systolic and/diastolic value) of the BP data and the contextual data (i.e. the question and the selected predetermined responses) and returning the notification level associated with those inputs.

The size of the correspondence database directly depends on the BP data (diastolic pressure, systolic pressure, and/or heart rate), the number of questions and the number of predetermined responses for each question.

The correspondence database 900 may be stored on the BP monitor 110a, the server 130 or the remote monitoring interface 120. The attribution of the notification level to the BP data using these BP data and the selected contextual data, may be carried out by the BP monitor 110a, the server 130 or the remote monitoring interface 120. In an embodiment, since the server 130 is on the pathway of the BP data and the selected contextual data from the BP monitor 110a to the remote monitoring interface 120, the attribution of the notification level is carried out by the server 130.

### Retake Feature

In some embodiments, depending on the selected contextual data, the BP monitor 110a may request the user to obtain another BP data. For example, when the selected contextual data is “have you rested for 5 min? No”, then the BP monitor 110a may request so, before starting another BP measurement.

### Health Condition

As presented previously, the BP monitor 110a may check a health condition for the BP data before displaying a question. In a further embodiment, the health condition is applied to other heath data of the user, collected by another measuring device of the RPM system 100, such as weight data measured by a body scale. For example, the RPM system 100 includes a body scale and the health condition is that there is a noticeable weight variation on two consecutive days.

### RPM Method

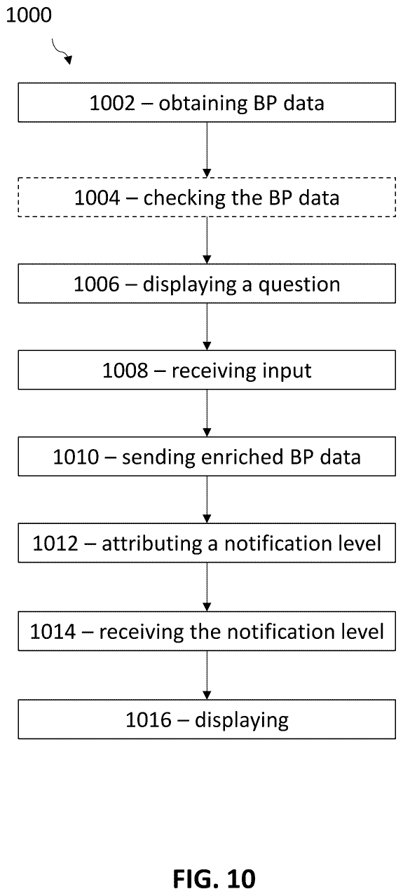

FIG. 10 shows a RPM method using the RPM system 100 disclosed previously. The steps are performed either by the BP monitor 110a, the remote monitoring interface 120 or the server 130.

At 1002, the BP monitor 110a obtains BP data using the BP sensor 206. The BP data is stored in the BP monitor 110a.

At 1004, in an embodiment, the BP monitor 110a checks whether the BP data verifies a health condition. The health condition may be a range for values of the BP data, as disclosed previously. For example, the heath condition is a high systolic value of the BP data (e.g., more than 140 mm Hg).

At 1006, in response to determining that the BP data verifies the health condition, or, in the absence of such check, after (or actually before, to create a rest time for the user before taking the measurement) obtaining the BP data 1002, the BP monitor 110a displays a question on the display, along with a plurality of predetermined responses. The predetermined responses can be selected by the user with the hardware input 210. The plurality of predetermined responses associated to the question form a plurality of contextual data.

In the absence of checking 1004, displaying the question 1006 may be performed before obtaining the BP data 1002. This slows down the BP measurement process for the user and increases the quality of the measurement.

At 1008, the BP monitors 110a receives a selected predetermined response, by the user by means of the hardware input 210. The selected predetermine response (example: “YES”) associated with the question (example: “have you taken your medication today?”) form a selected contextual data.

At 1010, the BP monitor 110a sends the BP data and the selected contextual data to the server 130 using the wireless communication module. The BP data and the selected contextual data form a bundle, as they are associated with one another. The BP data coupled to the selected contextual data may be called “enriched BP data”. In a variant, the enriched BP data may be sent directly from the BP monitor 110a to the remote monitoring interface 120.

At 1012, the server 130 attributes a notification level to the BP data using the enriched BP data, that is to say using the BP data and the selected contextual data. The attribution of the notification level 1012 is made amongst a plurality of available notification levels. More precisely, the attribution 1012 may be carried out using a query on a correspondence database 900 stored on the server 130, as disclosed previously. In a variant, the correspondence database 900 may be stored on the BP monitor 110a or the remote monitoring interface 120, and the attribution 1012 is carried out respectively by the BP monitor 110a or the remote monitoring interface 120.

At 1014, the remote monitoring interface 120 receives the enriched BP data with the attributed notification level.

At 1016, the remote monitoring interface 120 displays a display mode based on the attributed notification level.

In an embodiment, the display mode includes displaying the BP data or the enriched BP data with a specific layout associated with the label. For example, a red notification level entails a red font while a yellow notification level entails a yellow font.

In another embodiment, the display mode includes displaying a notification inbox, classified by the notification level.

In an embodiment, a display mode may include not displaying the BP data, notably when the notification level is minimum.

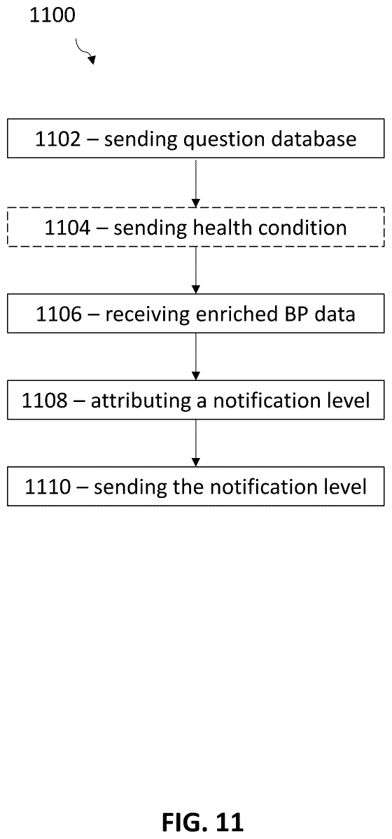

FIG. 11 shows a RPM method using the RPM system 100 disclosed previously from the server 130 stand point.

At 1102, the server 130 sends to the BP monitor 110a a question database, the question database including at least one question and a plurality of predetermined responses for each question. The question and the predetermined response form contextual data.

At 1104, in an embodiment, the server 130 sends to the BP monitor 110a a health condition. The health condition is to be checked with the BP data.

At 1106, the server 130 receives from the BP monitor BP data with a predetermined response of a question. The question and the selected predetermined response form selected contextual data.

At 1108, identical to step 1012, the server 130 attributes a notification level to the BP data. To that end, the server 130 stores a correspondence database as disclosed previously.

At 1110, the server 130 sends out to the remote monitoring interface the enriched BP data along with the attributed notification level.

### Additional Embodiments

In an alternative embodiment, the RPM system 100 includes the close monitoring interface of the smartphone 150 and the question is displayed on it, rather than the BP monitor 110a.

In another embodiment, the measuring device is replaced with a body scale 110b and the input hardware may include the weight sensor to select a response. Instead of monitoring the BP data, the RPM system here monitors the weight. Contextual data may involve lifestyle, meals, or response related to cardiac failure symptoms.

More generally, the physiological monitoring device comprises a user interface comprising a display and input hardware to receive a user input.

In another embodiment, the physiological measurement device 110 is a device capable of measuring temperature, blood oxygen saturation, lung or heart sounds, and/or capable of taking an electrocardiogram. An example of such device is given in document EP24217550. Particularly, this device includes a display and input hardware in a very similar fashion to that of BP monitor 110a.

In some embodiments, the functionalities of the BP monitor 110a, 200, the server 130, and/or the remote monitoring interface 120, 300 described herein are implemented, at least in part, by one or more processors executing instructions stored on one or more non-transitory computer-readable media. Such non-transitory computer-readable media may include, for example, semiconductor memories (e.g., flash memory, EEPROM, ROM, RAM), magnetic or optical storage media (e.g., hard disks, SSDs, CDs, DVDs), or any other physical storage device capable of storing executable program code and data structures. The memory 216 of the BP monitor 200 and the memory 406 of the server 400 may each comprise such a non-transitory computer-readable medium.

In an embodiment, a non-transitory computer-readable medium is provided in the BP monitor 110a, 200 storing instructions which, when executed by the processor 214 of the control unit 212, cause the BP monitor 110a, 200 to: obtain BP data using the BP sensor 206; display, on the display 208, one or more questions with a plurality of predetermined responses defining contextual data; receive, via the input hardware 210, a predetermined response selected by the user to form selected contextual data; form enriched BP data including the BP data and the selected contextual data; and transmit, via the wireless communication module 220, at least the enriched BP data toward the server 130 and/or the remote monitoring interface 120. In related embodiments, a non-transitory computer-readable medium provided in the server 130 stores instructions which, when executed by a processor 404 of the server 400, cause the server 130 to receive the enriched BP data, attribute a notification level using a correspondence database 900, 902 linking BP values and contextual data to notification levels or notification-level variations, and send to the remote monitoring interface 120 the enriched BP data together with the attributed notification level for display in a display mode determined by the notification level.

In another embodiment, a non-transitory computer-readable medium is provided in the monitoring device 302 storing instructions which, when executed by a processor 310 of the control unit 304, cause the remote monitoring interface 300 to receive the BP data, the selected contextual data and/or the attributed notification level from the server 130, and to present the BP data in one of a plurality of display modes corresponding to different notification levels, as described herein. These embodiments illustrate that the invention may be implemented as a computer-implemented system, a device including a programmed processor, and/or as a computer program product embodied in one or more non-transitory computer-readable media.

In view of the foregoing, the RPM system and associated methods described herein provide a concrete technical implementation that goes beyond the mere manipulation or display of abstract data. The system combines specific hardware components (including the BP monitor 110a with its BP sensor 206, user interface 204 and wireless communication module 220, the server 130, and the remote monitoring interface 120 running on the monitoring device 302) with dedicated software logic and data structures (including the question database and the correspondence database 900, 902) to generate enriched BP data and an attributed notification level in real time or near real time. The interaction between these hardware and software elements produces a technical effect in the operation of the RPM system 100, namely an improved way of capturing, transmitting, processing and presenting physiological measurements so as to reduce false positives and focus the professional's attention on clinically significant situations.

The result of the method implemented by the RPM system 100 is not limited to a mere numerical transformation of data, but is specifically configured to support medical decision-making and diagnostic assessment. By enriching the BP data with contextual data acquired at the time of measurement, and by mapping the enriched BP data to one of a plurality of notification levels using the correspondence database 900, 902, the system produces an output that is directly usable by a healthcare professional to determine whether additional diagnostic tests are warranted, whether the patient's treatment regimen should be adjusted, or whether an in-person consultation or emergency intervention is required. In practice, the notification level and associated display mode at the remote monitoring interface 120 thus constitute a clinically actionable result that can be used as a diagnostic aid, improving the reliability and interpretability of remote BP measurements.

The invention therefore addresses a concrete technical problem arising in remote patient monitoring systems, namely that high volumes of uncontextualized BP measurements generate numerous alerts and potential false positives that are difficult for professionals to triage efficiently. Prior-art RPM systems that merely forward raw BP values, or that rely on separate, non-integrated patient surveys, do not adequately distinguish between transient, behavior-related BP elevations and persistently abnormal values that reflect an underlying pathological condition. By integrating the acquisition of contextual data into the measurement workflow at the BP monitor 110a itself, and by using the correspondence database 900, 902 to automatically adjust the notification level and display mode based on both the BP data and the contextual data, the present RPM system 100 technically improves the functioning of the remote monitoring infrastructure and reduces the cognitive and operational burden on the professional.

Furthermore, the architecture disclosed herein is adaptable and scalable in practice. The question database and the correspondence database may be configured and updated over time by the professional, or by an institution, through the remote monitoring interface 120, enabling the RPM system 100 to be tailored to different patient populations, diseases, and clinical protocols without changing the underlying hardware. The described techniques may be implemented by one or more processors executing instructions stored on one or more non-transitory computer-readable media within the BP monitor 110a, the server 130 and/or the monitoring device 302, thereby providing a practical and repeatable technical implementation. The combination of specific hardware elements, structured data models for contextual and correspondence information, and automated notification-level attribution yields an overall improvement in the operation of remote patient monitoring technology.

In further embodiments, the RPM system 100 performs one or more additional actions after the display mode is generated at the remote monitoring interface 120. These actions may include initiating a clinical workflow, generating a physical or electronic output, or triggering an operational process.

In an embodiment, the remote monitoring interface 120 automatically generates and transmits a notification message to the patient, such as a recommendation to retake a measurement, to rest for a period of time, to take a prescribed medication, or to schedule an appointment. The notification may be delivered via SMS, email, push notification to the mobile device 150, or other communication channels supported by the RPM system 100.

In another embodiment, the RPM system 100 triggers the creation of a clinical task within an electronic health record (EHR) system, such as generating an entry in the patient's chart, creating a follow-up task for a nurse or physician, or initiating a standardized clinical protocol. The remote monitoring interface 120 may communicate with the EHR system through an application programming interface (API), thus enabling automatic population of clinically relevant data into a regulated medical system.

In a further embodiment, the RPM system 100 operates to escalate the patient's care pathway based on the notification level. For example, the system may: (i) automatically place the patient into a higher-intensity monitoring cohort; (ii) request additional physiological measurements from the patient; (iii) initiate telemedicine session scheduling; or (iv) route the case to an on-call professional for immediate review. These operational steps directly affect the delivery of medical care and are performed using the technical components of the RPM system 100.

In still another embodiment, the RPM system 100 causes the BP monitor 110a to perform a particular hardware action in response to the notification level. For example, if contextual data indicates an unreliable BP measurement (e.g., lack of rest before the measurement), the BP monitor 110a may automatically initiate a new measurement cycle or adjust the inflation profile of the pump to improve measurement quality. Such actions represent concrete transformations tied to the physical operation of the device.

In yet another embodiment, the RPM system 100 triggers the generation of a diagnostic indicator or recommendation that is used by a healthcare professional to support or refine a diagnostic assessment. For instance, the remote monitoring interface 120 may display a trend graph annotated with notification levels, or may flag a pattern of elevated BP values despite appropriate contextual conditions, thereby prompting the professional to evaluate the user for hypertension, medication non-response, or other medical conditions. This constitutes a practical, medically relevant application of the enriched BP data generated by the RPM system.

Collectively, these additional steps demonstrate that the method performed by the RPM system 100 is not limited to abstract data analysis or information presentation, but instead is directed to a concrete technological implementation that produces real-world clinical effects, executes physical device actions, interacts with external medical systems, and supports diagnostic decision-making.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles “a” and “an” may be employed in connection with various elements and components, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A remote patient monitoring, RPM, system, comprising:
a blood pressure, BP, monitor, including
a case,
a user interface mounted on the case, comprising a display and an input hardware,
a blood pressure, BP, sensor configured to generate blood pressure data about the user,
a wireless communication module,
a remote monitoring interface, configured to run on a remote monitoring device, wherein the remote monitoring device and the BP monitor are two distinct devices,
wherein the BP monitor is configured to send data to the remote monitoring interface using the wireless communication module,
wherein the RPM system is configured to:
obtain BP data using the BP sensor of the BP monitor,
display on the display of the BP monitor a question with a plurality of predetermined responses, wherein the question and the plurality of predetermined responses form a plurality of contextual data,
receive a predetermined response selected by the user using the input hardware of the BP monitor, wherein the question and the selected predetermined response form selected contextual data,
attribute a notification level to the BP data using the BP data and the selected contextual data, wherein the notification level is chosen amongst a plurality of available notification levels,
display at the remote monitoring interface a display mode based on the attributed notification level.

2. The RPM system of claim 1, wherein the plurality of available notification levels includes between 3 and 5 notification levels.

3. The RPM system of claim 1, wherein the remote monitoring interface includes a plurality of display modes, wherein each display mode includes a dedicated layout corresponding to a notification level, such that the display modes display BP data, for which contextual data are available, differently in function of the notification level.

4. The RPM system of claim 1, wherein the display mode includes a notification inbox, updated based on the attributed notification level.

5. The RPM system of claim 1, wherein the BP data is an average of several BP values.

6. The RPM system of claim 1, wherein displaying the question is performed in response to determining that the BP data verify a health condition.

7. The RPM system of claim 6, wherein the health condition is editable via the remote monitoring interface.

8. The RPM system of claim 1, wherein the display mode includes displaying the BP data along with the selected contextual data.

9. The RPM system of claim 1, further including a server, wherein:
the wireless communication module of the BP monitor sends to the server the BP data and the selected contextual data,
the remote monitoring interface receives from the server the BP data, the selected contextual data and the attributed notification level.

10. The RPM system of claim 1, wherein attributing the notification level includes:
querying a correspondence database using the BP data and the selected contextual data, wherein the correspondence database links a plurality of notification levels or of notification level variations to ranges values for the BP data and the contextual data, wherein notification level variations are to be applied to a preexisting notification level depending only on the BP data.

11. The RPM system of claim 10, further including a server, wherein:
the wireless communication module of the BP monitor sends to the server the BP data and the selected contextual data,
the remote monitoring interface receives from the server the BP data, the selected contextual data and the attributed notification level,
wherein the correspondence database is stored on the server and the attribution is performed by the server.

12. The RPM system of claim 10, wherein the correspondence database is stored on the BP monitor and the attribution is performed by the BP monitor.

13. The RPM system of claim 10, further including a remote monitoring device configured to run the remote monitoring interface, wherein the correspondence database is stored in the remote monitoring device.

14. The RPM system of claim 10, wherein the correspondence database is editable via the remote monitoring interface.

15. The RPM system of claim 1, wherein the wireless communication module is a cellular module.

16. A remote patient monitoring method, using a RPM system of claim 1, the method comprising:
obtaining BP data using the BP sensor of the BP monitor,
display on the display a question with a plurality of predetermined responses, wherein the question and the predetermined response form predetermined contextual data,
receive a predetermined response selected by the user using the input hardware, wherein the question and the selected predetermined response form selected contextual data,
attribute a notification level to the BP data using the BP data and the selected contextual data, wherein the notification level is chosen amongst a plurality of available notification levels,
display at the remote monitoring interface a display mode based on the attributed notification level.

17. A remote patient monitoring system, comprising:
a blood pressure (BP) monitor comprising:
(a) a BP sensor configured to obtain BP data of a user;
(b) a user interface comprising a display and input hardware;
(c) a wireless communication module; and
(d) a control unit configured to:
(i) display on the display at least one question with a plurality of predetermined responses, the question and predetermined responses defining contextual data;
(ii) receive, via the input hardware, a predetermined response selected by the user, thereby generating selected contextual data associated with the BP data; and
(iii) transmit the BP data and the selected contextual data via the wireless communication module;
a server configured to receive the BP data and the selected contextual data, and to attribute a notification level to the BP data using a correspondence database linking notification levels or notification-level variations to ranges of BP values and contextual data; and
a remote monitoring interface executed on a remote monitoring device, the remote monitoring interface being configured to receive the BP data, the selected contextual data, and the attributed notification level from the server, and to display the BP data in a display mode selected based on the attributed notification level.

18. A blood pressure (BP) monitor configured for use in a remote patient monitoring system, the BP monitor comprising:
a case;
a BP sensor configured to obtain BP data of a user;
a user interface mounted on the case, the user interface comprising a display and input hardware;
a wireless communication module; and
a control unit comprising at least one processor and a memory storing instructions which, when executed by the at least one processor, cause the BP monitor to:
obtain BP data using the BP sensor;
display, on the display, at least one question together with a plurality of predetermined responses, the question and the plurality of predetermined responses defining contextual data;
receive, via the input hardware, a predetermined response selected by the user, thereby generating selected contextual data associated with the BP data;
form enriched BP data including the BP data and the selected contextual data; and
transmit, via the wireless communication module, at least the enriched BP data toward a remote monitoring interface executed on a remote monitoring device, the enriched BP data being usable by the remote monitoring interface to determine a notification level and a display mode for presentation to a healthcare professional.

19. A non-transitory computer-readable medium storing instructions that, when executed by at least one processor of a blood pressure (BP) monitor, cause the BP monitor to perform a method comprising:
obtaining BP data using a BP sensor of the BP monitor;
displaying, on a display of the BP monitor, at least one question together with a plurality of predetermined responses, the question and predetermined responses defining contextual data;
receiving, via input hardware of the BP monitor, a predetermined response selected by a user, thereby generating selected contextual data associated with the BP data;
forming enriched BP data including the BP data and the selected contextual data; and
transmitting, using a wireless communication module of the BP monitor, the enriched BP data toward a server or a remote monitoring interface configured to attribute a notification level to the BP data based on the enriched BP data and to display the BP data in a display mode corresponding to the notification level.
